(19) United States
(12) Patent Application Publication
(10) Pub. No.: US 2024/0067909 A1
(43) Pub. Date: Feb. 29, 2024

(54) METHODS AND SYSTEMS FOR PROPAGATING MICROORGANISMS FOR MITIGATING MYCOTOXIN CONTAMINATION, AND RELATED SYSTEMS AND METHODS

(71) Applicant: POET Research, Inc., Sioux Falls, SD (US)

(72) Inventors: Andrew Manning, Sioux Falls, SD (US); Ben Gerdes, Yankton, SD (US); Amber Jerke, Yankton, SD (US)

(21) Appl. No.: 18/454,368

(22) Filed: Aug. 23, 2023

Related U.S. Application Data
(60) Provisional application No. 63/400,230, filed on Aug. 23, 2022.

(51) Int. Cl. C12M 1/00

(52) U.S. Cl. CPC C12M 21/12; C12M 47/10

(57) ABSTRACT

Systems and methods for propagating at least one microorganism expressing one or more enzymes to mitigate mycotoxin contamination at a bioprocessing facility.

### CROSS-REFERENCE TO RELATED APPLICATION

This nonprovisional patent application claims the benefit of U.S. Provisional Patent Application Ser. No. 63/400,230, filed on Aug. 23, 2022, wherein said provisional patent application is incorporated herein by reference in its entirety.

### BACKGROUND

There is a continuing need for systems and methods for mitigating mycotoxin contamination in process compositions of a bioprocessing facility.

### SUMMARY

The present disclosure includes embodiments of a system for propagating at least one microorganism expressing one or more enzymes to mitigate mycotoxin contamination. The system includes one or more propagation tanks. The system is configured to propagate the at least one microorganism and form a propagated composition including the at least one microorganism expressing one or more enzymes to mitigate mycotoxin contamination. The system is configured to couple to one or more points of a process in a bioprocessing facility to inoculate at least a portion of the propagated composition and/or a composition derived from the propagated composition into one or more process compositions to mitigate mycotoxin contamination. The bioprocessing facility is configured to produce one or more bioproducts from biomass.

The present disclosure includes embodiments of method of propagating at least one microorganism expressing one or more enzymes to mitigate mycotoxin contamination, wherein the method includes: propagating the at least one microorganism expressing one or more enzymes to mitigate mycotoxin contamination at a bioprocessing facility that produces one or more bioproducts from biomass, wherein propagating forms a propagated composition including the at least one microorganism expressing one or more enzymes to mitigate mycotoxin contamination; and inoculating at least a portion of the propagated composition and/or a composition derived from the propagated composition into one or more process compositions of the bioprocessing facility to mitigate mycotoxin contamination.

### BRIEF DESCRIPTION OF THE DRAWINGS

Various examples of the present disclosure will be discussed with reference to the appended drawings. These drawings depict only illustrative examples of the disclosure and are not to be considered limiting of its scope.

FIG. 1 is a process flow diagram of a non-limiting example of a system according to the present disclosure for propagating a microorganism expressing an enzyme to mitigate mycotoxin contamination;

FIG. 2 is a process flow diagram of another non-limiting example of a system according to the present disclosure for propagating a microorganism expressing an enzyme to mitigate mycotoxin contamination; and

FIG. 3 is a process flow diagram of a non-limiting example of a cereal grain-to-ethanol conversion process.

### DETAILED DESCRIPTION

The present disclosure relates to a systems and methods for propagating a microorganism that produces enzymes for mitigating mycotoxins present in biomass, where the propagation occurs at a bioprocessing facility that produces one or more bioproducts from the biomass.

In some embodiments, the system can be portable so that it can be transported to a point-of-use at one or more bioprocessing facilities and connect to one or more points in an overall process at a given bioprocessing facility. By co-locating such a system at a bioprocessing facility, mitigating mycotoxins in one or more process compositions can be performed according to a “just-in-time” protocol, if desired, on an as-needed basis to align with regional and/or seasonal variation in the presence of one or more mycotoxins.

Mycotoxin contamination can be variable and depend on various conditions including the weather. Some years, e.g., certain wet years, there may be widespread growth of the mycotoxin-producing fungi in crops resulting in serious mycotoxin contamination. Other years conditions may be such that there is no mycotoxin contamination. Mycotoxin contamination can also vary regionally. When mycotoxin contamination occurs it can be transported into bioprocessing facility processes via the biomass (e.g., grain such as corn). For example, in the context of a corn-ethanol process, mycotoxin contamination can become concentrated during processing. For example, as starch is consumed and fat is separated the mycotoxin ppm concentration in, e.g., dried distillers grains with solubles (DDGS), can rise above the acceptable threshold for one or more animal species that would normally consume DDGS in a ration. At these levels, DDGS prices may be discounted as the purchasers may have to treat or blend the DDGS to reduce the contamination to an acceptable level. Because mitigating mycotoxins may or may not be needed (e.g., due to seasonal and geographic variability) at any given time at a bioprocessing facility (in contrast to microorganisms that produce a bioproduct from biomass (e.g., ethanol produced by an ethanologen), it may be desirable to have a system according to the present disclosure be portable such as by mounting the system on a portable skid. For example, mycotoxin contamination can be determined on the feedstock (e.g., in-bound corn at a bioprocessing facility) so that a decision can be made whether to utilize a portable skid according to the present disclosure.

In some embodiments, a system according to the present disclosure for propagating a microorganism expressing an enzyme to mitigate mycotoxin contamination is configured to be movable as a single unit among two or more bioprocessing facilities. A bioprocessing facility includes facilities configured to produce one or more bioproducts such as biochemicals, biofuel, food, and feed (e.g., animal feed) from biomass using for example microorganisms such as bacteria and fungi. Non-limiting examples of bioproducts include one or more of alcohol (e.g., butanol, ethanol, and the like), carbon dioxide, protein (e.g., yeast protein, grain protein, and the like), pharmaceuticals, enzymes, fermented foods and drinks, distiller's grain (e.g., WDG, WDGS, DDG, DDGS), and oil. Non-limiting examples of biomass include many agricultural crops such as a “cellulosic” material (e.g., grain kernel fiber, crop waste (e.g., corn stover), wood, municipal waste, etc.) and “starchy” cereal grains (e.g., one or more of corn, barley, rye, sorghum, triticale, and wheat). In some embodiments, a system according to the present disclosure can be made to be portable to facilitate deployment at a bioprocessing facility when needed. For example, a system could be mounted on a skid so that a number of skid systems less than the total number of bioprocessing facilities in a service region could share skid systems as needed. A skid-mounted system can include decking, piping, valving, electrical connections, and the like to permit coupling to a bioprocessing facility as described herein. The decking can be configured for mobility. For example, the decking can be adapted for transport by a forklift. As another example, casters can be mounted to the decking. Alternatively, a skid system is a convenient package of unit operations that could be permanently sited at each bioprocessing facility, if desired. In some embodiments, a permanently sited skid system could serve as a “hub” for one or more other bioprocessing facilities located within a region (discussed below).

A system according to the present disclosure can one or more propagation tanks and is configured to propagate the microorganism and form a propagated composition that includes the microorganism expressing an enzyme to mitigate mycotoxin contamination. The number and size of propagation tanks can be selected on a number of factors such as whether two or more different propagation protocols will be used (e.g., different microorganisms and/or propagation mediums), process control, portability, and the like. In some embodiments, system according to the present disclosure can include 1 or more propagation tanks, 2 or more propagation tanks, 3 or more propagation tanks, or even 4 or more propagation tanks. In some embodiments, system according to the present disclosure can include 4 or less propagation tanks, 3 or less propagation tanks, or even 2 or less propagation tanks. In some embodiments, a propagation tank can have a volumetric capacity of 10,000 gallons or less, 5,000 gallons or less, 3,000 gallons or less, or even 2000 gallons or less. In some embodiments, a propagation tank can have a volumetric capacity in the range from 10 to 5,000 gallons, 50 gallons to 3,000 gallons from 100 to 2,500 gallons, or even from 100 to 500 gallons. In some embodiments, the size of a propagation tank can facilitate propagating a microorganism expressing an enzyme to mitigate mycotoxin contamination on an as-needed basis (“just-in-time”) and/or scaling up (e.g., from a pilot facility to a commercial facility). For example, propagation tank having a relatively smaller size (e.g., 3000 gallons or less) can have a relatively shorter time period for producing a propagated composition on a just-in-time basis. Also, having relatively smaller volumes of propagated composition can facilitate temperature control that may be used if the propagated composition is subsequent exposed to lysis (discussed below), which can facilitate quality control.

The propagated composition outputs of two or more propagation tanks can be coupled together and in fluid communication with one or more downstream processes (either on the portable skid or at the bioprocessing facility). Optionally, the propagated composition output of at least one propagation tank is not coupled to the propagated composition output of any other propagation output before being transferred to downstream processes. Two or more propagation tanks can be operated relative to each other to propagate a microorganism in any desired manner. For example, two or more propagation tanks can be operated relative to each other serially, in parallel, staggered, temporarily staged, etc. In addition, a given propagation tank can be operated in any desired manner such as continuous (continuous feed and draw), batch, fed-batch, and the like.

A propagation tank refers to a bioreactor adapted or configured to expose a microorganism in a propagation medium to one or more conditions such as pH, time, temperature, aeration, stirring and the like and form a propagated composition (e.g., a propagation broth). In some embodiments, relatively smaller tanks are more easily controlled with respect to one or more of the above-mentioned conditions.

With respect to aeration, one or more techniques can be used to introduce a gas into and aerate the propagation medium. For example, a gas can be introduced into the headspace of propagation tank at a pressure sufficient so that the gas diffuses into the propagation medium. As another example, a gas can be sparged into the propagation medium at a rate high enough so that the gas bubbles up and through the propagation medium and oxygen transfers into the propagation medium. Optionally, the propagation medium can be agitated or mixed to facilitate transferring oxygen into and throughout the propagation medium. For example, a continuous stirred tank reactor (CSTR) can be used to agitate or mix the propagation medium. The speed of the stirring mechanism (rpms) can be adjusted based on a variety of factors such as propagation tank size, viscosity of the propagation medium, and the like. As another example, a bubble column could be used to introduce gas (e.g., air and/or oxygen) into the propagation medium. In some embodiments, aeration can provide a dissolved oxygen concentration in a propagation medium in a range from greater than zero to 20 mg dissolved oxygen per liter of propagation medium, or even from 1 to 5 mg dissolved oxygen per liter of propagation medium as measured at the propagation temperature mentioned below. Alternatively, propagation could be performed anaerobically, which may result in larger propagation tanks to achieve similar production rates as aerobic propagation.

With respect to stirring, one or more techniques can be used to keep the contents of a propagation tank well mixed. Non-limiting examples of a stirring mechanism include a top entry agitator, recirculation pump, gas-pulsing system, aeration, large bubble mixing (pulsair), side entry agitator, combinations thereof, and the like.

The pH of the propagation medium can be at a pH that helps reproduce (propagate) and generate a desired population of microorganism within a desired amount of time. In some embodiments, the pH is between 4 and 7, between 4.5 and 7, or even between 4.5 and 6. Techniques for adjusting and maintaining pH of a propagation medium for propagating microorganisms include, e.g., adding one or more acidic materials and/or adding one or more basic materials.

With respect to temperature and time, the contents of a propagation tank (the propagation medium) can be maintained at temperature for time period to propagate a microorganism to a desired population size. In some embodiments, the temperature of a propagation medium can be controlled to a temperature in a range from 40° F. to 140° F., from 60° F. to 120° F., or even from 70° F. to 100° F. In some embodiments, propagation can occur for a time period in a range from greater than zero to 72 hours, from 1 hour to 48 hours, from 2 hours to 48 hours, or even from 10 hours to 30 hours.

Propagation tanks can be used to propagate the same or different microorganism. In some embodiments, two or more propagation tanks can used to propagate the same microorganism expressing an enzyme to mitigate mycotoxin contamination. In some embodiments, at least one propagation tank can be used to propagate two or more microorganisms expressing an enzyme to mitigate mycotoxin contamination that are different from each other (referred to as co-culture). In some embodiments, at least one propagation tank can be used to propagate a microorganism that is different from the microorganism that is propagated in the remaining propagation tank or tanks. For example, one microorganism can be a first strain of a microorganism that expresses one or more enzymes, and a second microorganism can be a second strain of the microorganism that expresses one or more enzymes that are different from at least one of the enzymes expressed by the first strain. As another example, one microorganism can be a yeast that expresses one or more enzymes, and a second microorganism can be a bacterium that expresses one or more enzymes that are different from at least one of the enzymes expressed by the yeast. Using different propagation tanks for microorganisms that express different enzymes can facilitate reliability and/or quality control, which can help meet final product (e.g., DDGS) specifications.

By propagating at least one a microorganism that is different from the others permits an “enzyme cocktail” to be produced, if desired. In some embodiments, growing different microorganism separately in different propagation tanks can make it easier to provide optimal growing conditions for different microorganisms and relatively better control of the proportionality of the various enzymes when combined to form an enzyme cocktail. In some embodiments, at least a portion of the propagated compositions of two or more propagation tanks can be combined in any desired ratio to make the enzyme cocktail. For example, a system according to the present disclosure can be configured to remove and adjustably combine at least a portion of the contents of two propagation tanks in a volumetric ratio of 1:1 or different than 1:1. Any desirable ratio can be selected based on one or more factors such as the number of propagation tanks and the total amount of protein (protein abundance) of a propagated composition. Changing the volumetric ratio can impact activity of the overall enzyme cocktail.

A wide variety of microorganisms and enzymes expressed by a microorganism to mitigate mycotoxin contamination can be produced using a propagation system according to the present disclosure. Likewise, a wide variety of mycotoxins can be mitigated depending on the one or more enzymes that are expressed by a microorganism. An enzyme can mitigate mycotoxin contamination by catalyzing the conversion of the mycotoxin to a less toxic form.

Non-limiting examples of mycotoxins that can be mitigated are chosen from aflatoxin, ochratoxin A, patulin, T-2, HT-2, beauvericin, neosolaniol, nivalenol, deoxynivalenol (DON), 3-ADON, 15-ADON, trichothecene, ochratoxin, zearalenone, and combinations thereof.

Non-limiting examples of enzymes can be used to mitigate mycotoxins are chosen from aflatoxin oxidase, 3-O acetyltransferase, peroxidase, F420H2-dependent reductases, Ery4, UDP-glycosyltransferase, laccase, manganese peroxidase, deoxynivalenol hydroxylase, lactono hydrolase, xylanase, peroxidase, 2cys-peroxiredoxin, and combinations thereof. In some embodiments, the enzyme can be a combination of enzymes, for example, an enzyme system or cocktail that works in two or more steps to detoxify a mycotoxin. For example, the enzyme system can be DepA/DepB, where DepA converts DON to 3-keto-DON and DepB, an NADPH dependent dehydrogenase, catalyzes the reduction of 3-keto-DON to 3-epi-DON.

Non-limiting examples of a microorganism expressing an enzyme to mitigate mycotoxin contamination and that can be propagated in s system according to the present disclosure are described in U.S. Pub. No. 2021/0403842 (Manning et al.), the entirety of which is incorporated herein by reference.

A propagation medium can include at least water, one or more carbon sources, and one or more nutrient sources for microorganism growth and reproduction.

As used herein, a “carbon source” refers to one or more compounds that include at least one carbon atom and can be used by a microorganism such as yeast to grow and/or reproduce to create additional biomass. Exemplary carbon sources include monosaccharides such as dextrose, fructose, galactose, mannose, xylose and the like; disaccharides such as lactose, maltose, sucrose, cellobiose and the like; oligosaccharides; polysaccharides such as cellulose, hemicelluloses, starch, xylene, and the like; single carbon substrates including only one carbon atom such as methanol; and polyols such as glycerol, but not limited thereto.

In addition to a carbon source, a non-carbon source or nutrient source may also be included to help propagate microorganisms. As used herein, a “nutrient source” refers to one or more materials or nutrients that can be used by a microorganism to grow and/or reproduce to create additional microorganisms and is different from a carbon source. In some embodiments, the nutrient source can be used as a carbon source. Nutrient sources and carbon sources can be obtained from a bioprocessing facility and/or transported to the bioprocessing facility from a supplier. One or more propagation tanks can be coupled to one or more sources to receive the contents therefrom and form a propagation medium (e.g., at least one source that provides water, nutrients, and carbon for the microorganism to propagate). In some embodiments, a system according to the present disclosure can be coupled to and in fluid communication with one or more points of a process in a bioprocessing facility to receive at least a portion of a process composition for use as a nutrient source and/or a carbon source. For example, at least one of the propagation tanks is configured to couple to one or more one or more points in the bioprocessing facility to receive at least a portion of a process composition. In some embodiments, using one or more process compositions from a bioprocessing facility can help condition the microorganism, if needed, reduce carbon-dioxide emissions by eliminating the need to transport such sources to the bioprocessing facility, and the like. Process compositions from a bioprocessing facility include process compositions present upstream from fermentation and/or downstream from fermentation. Non-limiting examples of process compositions from a bioprocessing facility include sugar compositions (e.g., sugar slip stream), starch compositions (e.g., starch slip stream), stillage compositions, and the like. Sugar slip streams are described in U.S. Pat. No. 10,233,466 (Redford), the entirety of which is incorporated herein by reference. Starch slip streams are described in U.S. Pat. No. 10,793,879 (Redford et al.), the entirety of which is incorporated herein by reference.

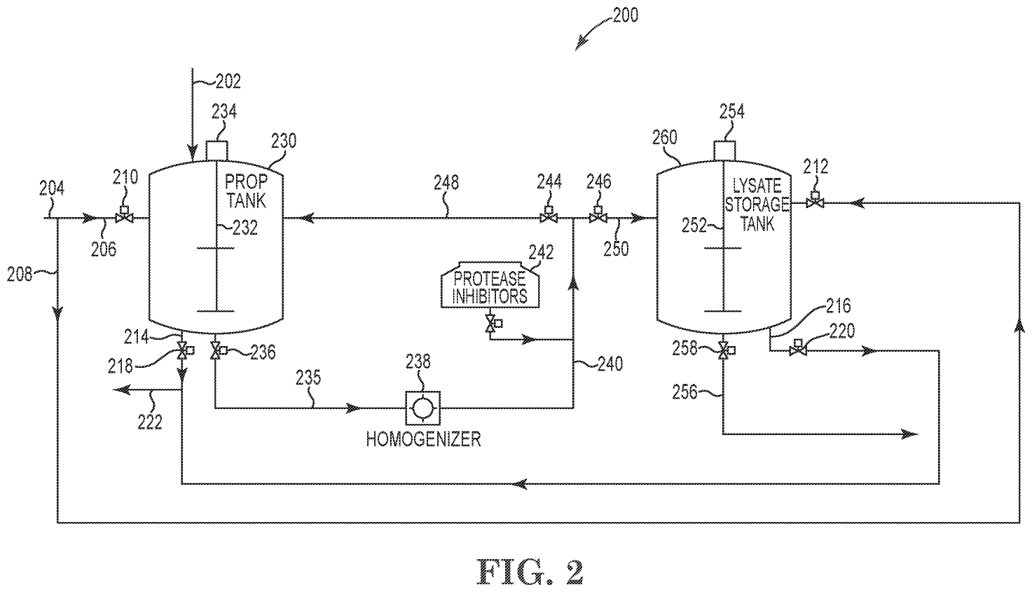

As used herein, a “stillage composition” refers to a back-end composition of a fermentation process after separating (e.g., via distillation) one or more bioproducts from beer to form at least one target bioproduct stream (e.g., ethanol) and one or more co-product streams (e.g., whole stillage). A stillage composition can include whole stillage, at least one stillage composition derived from whole stillage, and combinations thereof. In some embodiments, whole stillage is derived from distilling beer in a dry-grind corn ethanol biorefinery. Non-limiting examples of a stillage composition derived from whole stillage include thin stillage, concentrated thin stillage (syrup), defatted syrup, defatted emulsion, clarified thin stillage, distiller's oil, distiller's grain, distiller's yeast, and the like. Defatted syrup and defatted emulsion are examples of stillage compositions that remain after fat (e.g., corn oil) has been separated from syrup and emulsion, respectively, and can be referred to as “defatted stillage compositions.” Non-limiting illustrations showing one or more of the above-mentioned stillage compositions are shown in each of FIGS. 2-5B (discussed below). Non-limiting examples of methods and systems for processing stillage streams are also described in U.S. Pat. No. 8,702,819(Bootsma); U.S. Pat. No. 9,061,987(Bootsma); U.S. Pat. No. 9,290,728(Bootsma); U.S. Pat. No. 10,059,966 (Bootsma); U.S. Pat. No. 11,248,197 (Bootsma); and U.S. Pub. No. 2020/0140899 (Bootsma); wherein the entirety of each of said patent documents is incorporated herein by reference. A stillage composition also includes back-end compositions of a fermentation process after separating one or more bioproducts from beer using separation technologies other than distillation (e.g., membrane separation, gas stripping, absorption).

Optionally, one or more inducers may be present to promote enzyme production. Non-limiting examples of inducers include IPTG (isopropyl B-D-1-thiogalatopyranoside), galactose, glucose, and combinations thereof. Also, in some embodiments, temperature can be selected to induce enzyme production. Alternatively, inducers can be avoided if desired. For example, a constitutive promoter that continuously produces enzymes throughout the lifecycle of the microorganism can be used such that an inducer may not be needed.

Optionally, one or more co-factors may be present for microorganism growth, and/or for enzyme production and/or function post-lysis to mitigate mycotoxin contamination. Non-limiting examples of co-factors include iron chloride, 5-aminolevulinic acid, NADH (nicotinamide adenine dinucleotide), and combinations thereof.

When propagation in a propagation tank is complete the propagated composition can be inoculated as-is into one or more process compositions at a bioprocessing facility to mitigate mycotoxin contamination, and/or be further processed to form a composition derived from the propagated composition prior to inoculation. As used herein, a propagated composition can also be referred to as a propagation broth or whole propagation broth that is formed in a propagation tank. In some embodiments, directly inoculating a fermenter in a commercial bioprocessing facility with a whole propagation broth from one or more propagation tanks can facilitate a just-in-time protocol. As discussed below, examples of a composition derived from a propagated composition/broth include a concentrated propagation broth, a lysate, a concentrated lysate, enzyme that is isolated from a propagation broth, enzyme that is isolated from a lysate, and combinations thereof.

In some embodiments, after the desired growth is obtained the culture can be cooled, to help settle the microorganism to the bottom of the propagation tank, where excess liquid can be removed to concentrate the microorganism prior to lysis of the microorganism. In some embodiments, the excess liquid can be removed via using gravity without a centrifuge (e.g., sedimentation), which can be energy efficient and maintenance friendly.

Optionally, a propagation broth can be exposed to a lysis system to form a lysate. A lysate is formed by lysing the microorganism that expresses the mycotoxin mitigating enzyme. Lysis of a microorganism refers cellular disruption that results in the outer boundary or cell membrane being broken down or destroyed in order to release inter-cellular materials. Lysing a microorganism can release mycotoxin mitigating enzyme making the enzyme relatively more available to mitigate mycotoxin in a process composition in a bioprocessing facility. In addition, lysing a microorganism essentially kills the microorganism, which advantageously helps avoid competition (e.g., competition consuming carbon source such as sugar) between the microorganism which expresses the mycotoxin mitigating enzyme and any other microorganism (e.g., ethanologen) used in the bioprocessing facility to make one or more bioproducts from biomass. In some embodiments, a microorganism may release enzyme such that a lysis system may not be used.

A variety of techniques can be used in a lysis system for lysing a microorganism. Methods of lysis are available on both a macro and micro scale. Lysis can be performed using one or more mechanical methods, one or more non-mechanical methods, and combinations thereof. In some embodiments, a recycle loop can be used to recycle the output of a lysis operation and repeatedly expose to the lysis operation until a fully lysed product is achieved.

In some embodiments, as mentioned above, the contents of a propagation tank can be cooled and/or concentrated prior to lysing. Further, the temperature can be controlled throughout lysis so that the temperature does not go above a temperature (below the melting point of the enzyme) that inactivates the enzymes to an undue degree, thereby resulting in an ineffective lysate. Similarly, the temperature of the enzyme can be controlled to be low enough during any other process or storage that occurs between propagation and inoculation to avoid inactivating the enzymes to an undue degree. For example, a temperature control system could be included that maintains a lysate in a frozen condition until use. In some embodiments, the system is configured to control the temperature of the enzyme present in the system to be 140° F. or less, 130° F. or less, 120° F. or less, 110° F. or less, or even 100° F. or less. A system according to the present disclosure can be configured in a variety of ways to control the temperature throughout propagation and/or lysis as described. In some embodiments, a portable skid can include fluid-jacketed vessels that are connected to one or more of a cooling tower water, heat exchanger, or one or more other temperature-controlled water sources at a bioprocessing facility.

Non-limiting examples of mechanical lysing include using homogenizers, bead mills and the like. A homogenizer such as a high-pressure homogenizer (HPH) is an example of equipment for microbial disruption. In an HPH, cells in media are forced through an orifice valve using high pressure causing disruption of the cell membrane due to high shear force at the orifice when the cell is subjected to compression while entering the orifice and expansion upon discharge. Lysis with an HPH can involve passing microorganisms through an HPH one or more times depending on the pressure selected and/or microorganism involved. For example, in some embodiments, yeast can be passed through an HPH at a pressure from 1000 to 1200 bar to cause a sufficient degree of lysis. In some embodiments, bacteria can be lysed at lower pressures. As another example, in some embodiments, microorganisms can be passed through an HPH at a pressure less than 1000 bar (e.g., from 650 to less than 1000 bar) multiple times to cause a sufficient degree of lysis.

A bead mill involves disrupting cells by agitating tiny beads made of glass, steel, or ceramic along with a cell suspension at high speeds. The beads collide with the cells and break open the cell membrane and releasing the intracellular components by shear force.

Non-mechanical lysing methods include one or more physical methods of lysing, one or more chemical methods of lysing, and one or more biological methods of lysing.

As used herein, physical lysing refers to non-contact methods that utilize one or more external forces such as heat, pressure, and sound energy, to rupture the cell membrane. Non-limiting examples of physical lysing include thermal lysis, cavitation, osmotic shock, and the like. Thermal lysis involves repeated freezing and thawing cycles, which cause formation of ice on the cell membrane and helps break down the cell membrane. Cavitation involves the formation and subsequent rupture of cavities or bubbles. Such cavities can be formed by reducing the local pressure which can be done by increasing the velocity, ultrasonic vibration, and the like. Reduction of pressure causes the collapse of the cavity and a large amount of mechanical energy to be released in the form of a shockwave that propagates through the medium. The high energy of the shock wave can disintegrate the cell membrane. Ultrasonic sound and hydrodynamic methods can be used for generating cavitation used to disrupt cells.

Chemical lysing refers to using lysis buffers to disrupt the cell membrane. Non-limiting examples of chemical lysing include alkaline lysis, and detergent lysis. Alkaline lysis can break the cell membrane by changing the pH. Detergents can be added in detergent lysis to solubilize the membrane proteins and to rupture the cell membrane.

Non-limiting examples of biological lysing include using one or more enzymes such as lysozyme, lysostaphin, zymolase, cellulose, protease or glycanase.

With respect to a system according to the present disclosure, a lysis system can be coupled directly or indirectly to one or more of propagation tanks and configured to form a lysate with at least a portion of contents from the one or more propagation tanks. In some embodiments, two or more propagation tanks can be multiplexed to a single lysis system such that they are temporally staged and cycled to the lysis system in a manner to produce a lysate. In some embodiments, multiplexing propagation tanks with a single lysis system permits a lysate to be produced in a just-in-time manner for sequential fermentations.

A system according to the present disclosure that includes a lysis system can tailor the lysis system to use one or more lysis techniques in a manner that facilitates just-in-time delivery of the lysate and/or permits a relatively simple method of providing the lysate. For example, in some embodiments, a lysis system could include one or more lysis techniques that are relatively gentle and/or avoid additional processing of the lysate prior to inoculating a process composition with the lysate. Non-limiting examples of lysis techniques that are relatively gentle and/or avoid additional processing of the lysate include mechanically and/or physically lysing a microorganism to form a lysate of the microorganism.

In some embodiments, lysing systems and methods according to the present disclosure do not include chemically lysing and/or biological lysing. For example, not using chemical lysing can avoid having to subsequently process the lysate to alter one or more properties prior to inoculating a process composition. For example, in some enzyme facilities, more aggressive lysing techniques use chemical lysing agents. The lysate is then typically processed to remove components that would be detrimental to, e.g., fermentation.

Optionally, a system according to the present disclosure can include (be coupled to and in fluid communication with) one or more enzyme stabilizers and/or one or more protease inhibitors. The system can be configured to combine the one or more protease inhibitors and/or one or more stabilizers with the propagated composition and/or a composition derived from the propagated composition at one or more points in the system. A protease inhibitor includes one or more chemicals that block or inhibit the action of a corresponding protease (e.g., an endogenous protease), which once the organism is lysed will have access to the full array of protein from inside and outside the cell. In some embodiments, a protease inhibitor can be added during lysing and/or prior to storage in order to maintain the functionality of the enzyme that mitigates mycotoxin and to not degrade it by native (endogenous) protease function of the microorganism. Non-limiting examples of a protease inhibitor includes 4-benzenesulfonyl fluoride hydrochloride (AEBSF), aprotinin, bestatin, trans-Epoxysuccinyl-L-leucylamido(4-guanidino)butane (E64), leupeptin, pepstatin A, and combinations thereof.

An enzyme stabilizer includes one or more chemicals that help maintain the functionality of the enzyme that mitigates mycotoxin, especially while the enzyme is present in the lysate. In some embodiments, a stabilizer can be added during lysing and/or prior to storage in order to maintain the functionality of the enzyme that mitigates mycotoxin. Non-limiting examples of a stabilizer includes glycerol, one or more sugars, one or more dextrans, polyethylene glycol, one or more amino acids and derivatives thereof, one or more salts, one or more surfactants, and combinations thereof.

Because a system according to the present disclosure can be co-located at a bioprocessing facility, transportation to the bioprocessing facility can be eliminated so that little or no protease inhibition and/or stabilization is necessary, especially if propagating a microorganism that produces enzymes for mitigating mycotoxins is performed according to a just-in-time protocol. In some embodiments, a system according to the present disclosure that is located at a bioprocessing facility can service as a “hub” to one or more other bioprocessing facilities located within a relatively local region. For example, if a bioprocessing facility located within a relatively local region needs a relatively small dose for each fermentation reactor it may make economic sense to utilize a system according to the present disclosure that is located at another bioprocessing facility. In some embodiments, a relatively small dose can include 150 gallons or less, 100 gallons or less, or even 50 gallons or less for each fermenter. Containers having corresponding capacities can be filled and transported to one or more other bioprocessing facilities located within a relatively local region.

In some embodiments, if needed, the optional enzyme stabilizers and/or protease inhibitors are a simple short-term input (relatively less inhibitor and/or stabilizer) because long term storage can be avoided. In some embodiments, the system is configured to combine the one or more protease inhibitors with a lysate at a weight ratio in a range from 1:10,000 to 1:10, from 1:10,000 to 1:500, or even from 1:10,000 to 1:1,000. In some embodiments, the system is configured to combine one or more enzyme stabilizers such as glycerol with the propagated composition and/or a composition derived from the propagated composition in an amount from greater than zero to 15 weight percent.

Optionally, a system according to the present disclosure can include (be coupled to and in fluid communication with) a system configured to separate and/or concentrate enzyme from a propagation composition or a composition derived from the propagation composition (e.g., a lysate). Concentrating the enzyme may help meet “generally recognized as safe” (GRAS) requirements. In some embodiments, a filtration system can be configured to separate and concentrate enzyme from lysate debris. A non-limiting example of a filtration system could include two or more stages of progressively smaller filter openings to separate and concentrate enzyme from the lysate. For example, a filtration system could include a first-stage filter having a membrane with a molecular cut off of 100 K Daltons so that when lysate is filtered with the membrane free soluble protein can flow through the membrane to form a permeate while most cell debris remains in the retentate. The filtration system could include a second-stage filter having a membrane with a 10 K Da membrane so that when permeate from the first-stage filter is filtered with the membrane the retentate would contain the enzymes of interest in a concentrated form, while the permeate would include mainly soluble small proteins, minerals, and/or the like.

Optionally, a system according to the present disclosure can include (be coupled to and in fluid communication with) a storage system to temporarily store a propagation composition or a composition derived from the propagation composition. A storage system can facilitate forming an enzyme cocktail by using a single storage tank to combine the contents of two or more propagation tanks, as described herein, and/or temporarily stage the propagation composition or a composition derived from the propagation composition prior to inoculating a process composition in the bioprocessing facility. If desired, a dedicated storage tank could be used for each propagation tank.

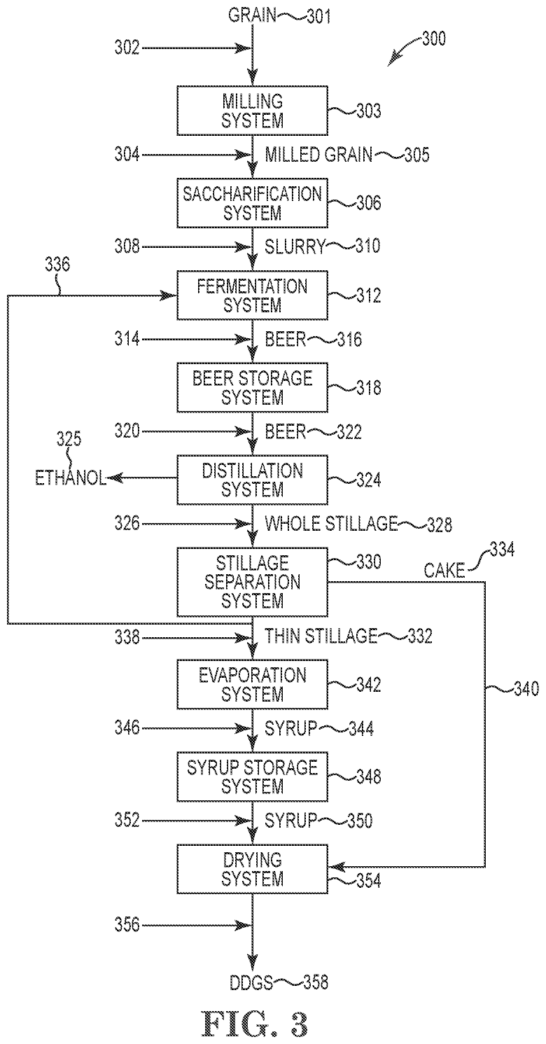

A system according to the present disclosure can be coupled to and in fluid communication with one or more points of a process in a bioprocessing facility to inoculate at least a portion of the propagated composition and/or a composition derived from the propagated composition (e.g., a lysate) into one or more process compositions at a bioprocessing facility to mitigate mycotoxin contamination. Non-limiting examples of process compositions at a bioprocessing facility include a feedstock composition, a milling composition, a steeping composition, a propagation composition (e.g., including an ethanologen), a saccharification composition, a fermentation composition, a stillage composition (see above), and combinations thereof. For example, a propagated composition including microorganism that produces enzymes for mitigating mycotoxins could be introduced to a fermentable composition that also includes a microorganism that ferments sugar to produce a bioproduct such as ethanol (also referred to co-culture or chimeric fermentation). Non-limiting examples of such process compositions at a bioprocessing facility are further discussed below in connection with FIG. 3.

A system according to the present disclosure can include a control system that includes one or more controllers in electrical communication with one or more sensors, valves, switches, and the like. The controller can transmit and receive signals during one or more processes at a bioprocessing facility and execute program instructions to prepare a propagated composition or composition derived therefrom and deliver it to one or more process compositions in the bioprocessing facility.

In some embodiments, it can be determined if mycotoxin is present in incoming grain and/or one or more process compositions in a bioprocessing facility, and then produce a propagated composition or composition derived therefrom using a system according to the present disclosure if mycotoxin is determined to be present. If mycotoxin is not determined to be present, the enzyme production system according to the present disclosure can be idled, turned off, or sent to another bioprocessing facility where mycotoxin is determined to be present. For example, a concentration of one or more mycotoxins can be measured in incoming grain and/or one or more process compositions in a bioprocessing facility. A variety of methods can be used to detect the presence of or measure the concentration of a mycotoxin. For example, the presence of DON can be determined using, e.g., NEOGEN™ VERATOX™ for DON 2/3 kit, which is commercially available from Neogen® Corporation.

In some embodiments, mycotoxin levels may be monitored and communicated to a controller that can be used to control the propagation of a microorganism expressing an enzyme to mitigate the mycotoxin contamination. Mycotoxin levels may be monitored at any point in the bioprocessing facility. Alternatively, monitoring may be omitted and the propagation of a microorganism expressing an enzyme to mitigate the mycotoxin contamination may be conducted based on expected values of mycotoxins. At times when no toxin is present the propagation system can be idled to reduce costs. For example, grain may be tested prior to entry into the bioprocessing facility, and in crop years and regions where mycotoxins are determined to be present above a threshold value the propagation system can be employed.

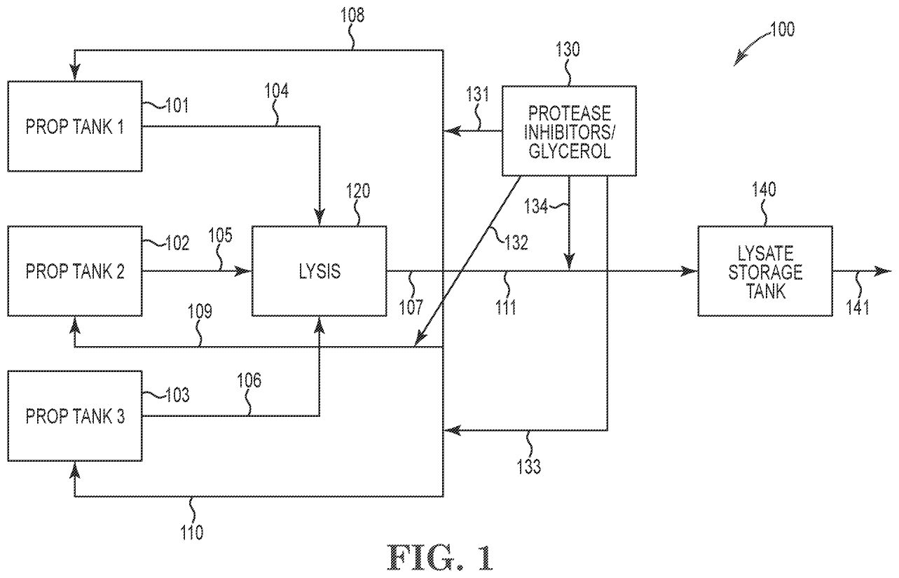

The discussion below makes reference to FIGS. 1-3. The schematic figures are for illustration purposes and are not necessarily drawn to scale.

FIG. 1 is a schematic illustrating a non-limiting embodiment of a system 100 for propagating one or more microorganisms expressing an enzyme to mitigate mycotoxin contamination. The system 100 includes three propagation tanks 101, 102, and 103. The system 100 is configured to propagate the microorganism in each propagation tank and form a propagated composition. The microorganism and/or the enzyme to mitigate mycotoxin contamination may the same or different among each of the propagation tanks 101, 102, and 103.

Lysis system 120 is coupled to each of the propagation tanks 101, 102, and 103 and receives contents of propagation tank 101 via stream 104, contents of propagation tank 102 via stream 105, and contents of propagation tank 103 via stream 106. The lysis system 120 is configured to expose the contents of each of the propagation tanks 101, 102, and 103, to one or more lysing techniques and form a lysate in stream 107. As shown, at least a portion of stream is recycled to each of the propagation tanks 101, 102, and 103, via streams 108, 109, and 110, respectively, while lysing. Also, as shown, one or more protease inhibitors 130 can be introduced into streams 108, 109, and 110 via streams 131, 132, and 133, respectively, while lysing. Further, one or more protease inhibitors 130 can be introduced into lysate stream 111 via stream 134 after lysing and prior to storage system 140.

As shown, the system 100 is configured to couple to inoculate at least a portion of the lysate from storage system 140 via stream 141 into one or more process compositions in a bioprocessing facility (e.g., into or upstream from a fermentation broth). Non-limiting examples of such one or more process compositions are further discussed below in connection with FIG. 3.

FIG. 2 is a process flow diagram illustrating a non-limiting embodiment of a system 200 for propagating microorganisms expressing an enzyme to mitigate mycotoxin contamination. The system 200 includes one propagation tank 230. One or more components 202 can be introduced into the propagation tank 230 to create a propagation medium while valve 236 is closed. For example, the one or more components can include an inoculum, nutrient source and/or carbon source (e.g., thin stillage), process water, and the like. The inoculum can include one or more microorganisms expressing an enzyme to mitigate mycotoxin contamination. The system 200 is configured to propagate the microorganism in the propagation tank 230 and form a propagated composition 235. Propagation tank 230 can include a stirring mechanism 232 driven by a motor 234. A homogenizer 238 is located downstream from the propagation tank 230 with a recycle line 248 returning homogenizer output 240 to the prop tank 230, while valve 246 is closed and valve 244 is open. The recycle loop 248 can be maintained as long as necessary to disrupt the microorganisms and produce an enzyme lysate 250. After the desired amount of recycle, valve 244 can be actuate closed and the valve 246 between the prop tank 230 and lysate storage tank 260 can be actuated open to allow the lysate 250 to flow to the storage tank 260 prior to being communicated to one or more process compositions in a bioprocessing facility (e.g., yeast propagation and/or fermentation vessels) via stream 256. Lysate storage tank 260 can include a stirring mechanism 252 driven by a motor 254.

This system 200 utilizes a relatively more gentle, mechanical lysing process via homogenizer 238 to avoid subsequent cleanup system that may be needed with a chemical lysing process. Chemical lysing includes adding one or more chemicals to help loosen or lyse the membrane of the microorganism. Non-limiting examples of such chemicals include one or more cell wall degrading enzymes and/or one or more chemicals that can dissolve the membrane/cell wall. Because the chemical added may have the same potential to lyse the microorganism (e.g., yeast) used in the in propagation and/or fermentation at the bioprocessing facility (e.g., for ethanol production) a lysate that is formed from chemical lysing may be “cleaned up” prior to being added to a downstream propagation and/or fermentation process. Otherwise, the presence of the chemical could impact propagation and/or fermentation to an undue degree (e.g., by causing fermentation to stall or never begin). If desired, separating chemical lysing compounds from a lysate could be performed using a filtration system as similarly discussed above with respect to isolating and concentrating the enzyme from the lysate. But, because of the potentially detrimental impact of the chemical downstream, the presence of any residual chemical after clean up may be monitored, which can increase the complexity and cost.

Also, the system 200 in FIG. 2 can avoid relatively long-term storage and transportation so that little or no stabilization is necessary.

Both propagation tank 230 and lysate storage tank 260 are jacketed vessels that permit a flow of temperature-controlled water to control the temperature of the contents. As shown in FIG. 3, a portion 206 of temperature-controlled water 204 can flow into the jacketed portion of propagation tank 230 while valve 210 is open, and a portion 208 of temperature-controlled water 204 can flow into the jacketed portion of lysate storage tank 260 while valve 212 is open. Also, water 214 can flow out of the jacketed portion of propagation tank 230 while valve 218 is open, and water 216 can flow out of the jacketed portion of lysate storage tank 260 while valve 220 is open. Streams 214 and 216 combine to form stream 222.

Protease inhibitors 242 can be introduced by actuating a valve open. Protease inhibitors are a simple short term stabilization input and can be added during lysing and/or after lysing.

FIG. 3 is a flow diagram of a cereal grain-to-ethanol conversion process depicting an ethanol biorefinery operation 300 that produces animal feed as a co-product. Grain 301 that has been contaminated with toxin is used as feedstock for the production of ethanol in the biorefinery. The grain 301 is reduced in size in a milling system 303. The milled grain 305 is mixed with water and further treated, for example thermally and/or enzymatically, to convert starch and fiber into fermentable sugars in a saccharification system 306. The resulting slurry 310 is combined with an ethanologen (e.g., yeast) to convert the sugars into ethanol in a fermentation system 312. In some embodiments, the saccharification and fermentation can occur simultaneously in a single system (e.g., in an SSF fermentation system). The fermentation product, or beer 322, includes ethanol, water, oil, dissolved solids, toxin, protein, yeast, and residual carbohydrates including starch, sugar, and fiber. The beer 316 may be collected in a beer storage system 318 prior to further processing. The beer 322 is distilled in a distillation system 324 to separate the ethanol 325 from the other components, called whole stillage 328, of the beer 322. The whole stillage 328 is processed in a stillage separation system 330 into cake 334 and thin stillage 332. The cake 334 contains more of the solid particulate matter from the beer 322 including fiber, protein, yeast, and residual solid starch and some liquid including water, oil, and dissolved solids. The thin stillage 332 contains more of the liquid from the beer 322 including water, oil, and dissolved solids. The toxin is distributed in the cake 334 and thin stillage 332. However, because the toxin is water soluble, more of it is contained in the thin stillage 332. The thin stillage 332 is concentrated in an evaporation system 342 to form syrup 344. The syrup 344 may be collected in a syrup storage system 348 prior to further processing. The syrup 350 and cake 334 may be combined and dried in a drying system 354 to produce dried distillers grains with solubles (DDGS) 358. Examples of biorefinery operations are described in U.S. Pat. Nos. 7,842,484, 8,409,640, 7,919,291, 8,470,550, 8,748,141, 8,679,793, 8,597,919, 8,702,819, 4,092,434, and 4,316,956 all of which are hereby incorporated by reference.

As described herein, at least a portion of the propagated composition and/or a composition derived from the propagated composition can be used to inoculate one or more process compositions to mitigate mycotoxin contamination. In the example of FIG. 3, a propagated composition (and/or a composition derived therefrom) can be introduced into one or more process composition of the biorefinery operation. Enzyme or enzymes that are introduced into a process composition react with the toxin (e.g., modify a ring structure of the toxin) to form a less toxic or non-toxic compound. Since the toxin enters the biorefinery with the grain, it is present in many of the biorefinery process streams and the a propagated composition (and/or a composition derived therefrom) may be introduced to any of these streams/locations to react with the toxin. For example, the a propagated composition (and/or a composition derived therefrom) may be introduced in a stream 302 that mixes with the grain 301 prior to or during milling; in a stream 304 that mixes with the milled grain 305 prior to or during saccharification 306; in a stream 308 that mixes with the slurry 310 prior to or during fermentation 312; in a stream 314 or 320 that mixes with the beer 316 or beer 322 prior to or during distillation 324; in a stream 326 that mixes with the whole stillage 328 prior to or during stillage separation 330; in a stream 338 that mixes with the thin stillage 332 prior to or during evaporation 342; in a stream 346 or 352 that mixes with the syrup 344 or syrup 350, respectively, prior to or during drying 354; at 340 with cake 334 prior to or during drying 354; and/or in a stream 356 that mixes with the DDGS 358.

While introduction in any one or more of the streams 302, 304, 308, 314, 320, 326, 338, 340, 346, 352, and 356 may remediate the toxin, according to the present disclosure, certain factors enhance the effectiveness of the remediation. It has been found that mixing in an aqueous environment, a relatively longer dwell time, and/or suitable enzymatic reaction conditions facilitate the reaction. Similarly, less volume of the propagated composition (and/or a composition derived therefrom) may be required if a stream is chosen in which the toxin has been concentrated. The toxin levels may be concentrated three to five times in DDGS in an ethanol operation. Most of the one or more toxins are concentrated via the thin stillage to syrup process stream. A portion of the thin stillage 336 is often used as recycled water that is fed back to a prior process step, such as saccharification 306 and/or fermentation 312.

Following are exemplary, numbered embodiments of the present disclosure:

1. A system for propagating at least one microorganism expressing one or more enzymes to mitigate mycotoxin contamination, wherein the system comprises one or more propagation tanks, wherein the system is configured to propagate the at least one microorganism and form a propagated composition comprising the at least one microorganism expressing one or more enzymes to mitigate mycotoxin contamination, and wherein the system is configured to couple to one or more points of a process in a bioprocessing facility to inoculate at least a portion of the propagated composition and/or a composition derived from the propagated composition into one or more process compositions to mitigate mycotoxin contamination, wherein the bioprocessing facility is configured to produce one or more bioproducts from biomass.

2. The system of embodiment 1, wherein the composition derived from the propagated composition is chosen from a concentrated propagation broth, a lysate of the at least one microorganism, a concentrated lysate of the at least one microorganism, enzyme that is isolated from a propagation broth of the at least one microorganism, enzyme that is isolated from a lysate of the at least one microorganism, and combinations thereof.

3. The system of any preceding embodiment, wherein the system is configured to be movable as a single unit among two or more bioprocessing facilities, wherein each bioprocessing facility is configured to produce one or more bioproducts from biomass.

4. The system of any preceding embodiment, wherein the each of the propagation tanks has a volumetric capacity of 10,000 gallons or less.

5. The system of any preceding embodiment, wherein the each of the propagation tanks has a volumetric capacity of 3,000 gallons or less.

6. The system of any preceding embodiment, wherein the one or more propagation tanks comprises two or more propagation tanks, wherein each propagation tank comprises at least one microorganism expressing one or more enzymes to mitigate mycotoxin contamination, and wherein at least one of the two or more propagation tanks comprises at least one microorganism that expresses one or more enzymes to mitigate mycotoxin contamination than is different from at least one microorganism that expresses one or more enzymes to mitigate mycotoxin contamination that is present in another propagation tank.

7. The system of embodiment 6, wherein the system is configured to remove and adjustably combine at least a portion of the contents of at least two propagation tanks in a volumetric ratio of 1:1 or different than 1:1.

8. The system of any preceding embodiment, wherein at least one of the propagation tanks is configured to couple to one or more one or more points in the bioprocessing facility to receive a composition chosen from a nutrient source, a carbon source, and combinations thereof.

9. The system of any preceding embodiment, further comprising a storage system in fluid communication with at least one of the propagation tanks, wherein the storage systems is configured to receive at least a portion of contents directly or indirectly from the at least one of the propagation tanks, wherein the storage system comprises one or more storage containers, wherein each storage container has a capacity of 150 gallons or less.

10. The system of any preceding embodiment, further comprising a source of one or more protease inhibitors, wherein the system is configured to combine the one or more protease inhibitors with the propagated composition and/or a composition derived from the propagated composition at one or more points in the system.

11. The system of embodiment 10, wherein the system is configured to combine the one or more protease inhibitors with lysate at a weight ratio in a range from 1:10,000 to 1:10.

12. The system of embodiment 10, wherein the system is configured to combine the one or more protease inhibitors with lysate at a weight ratio in a range from 1:10,000 to 1:1000.

13. The system of any preceding embodiment, further comprising a source of one or more stabilizers, wherein the system is configured to combine the one or more stabilizers with the propagated composition and/or a composition derived from the propagated composition at one or more points in the system.

14. The system of any preceding embodiment, further comprising a lysis system in fluid communication with one or more of the propagation tanks, wherein the lysis system is configured to form a lysate with at least a portion of propagated composition from the one or more propagation tanks.

15. The system of any preceding embodiment, wherein the system is configured to control the temperature of the enzyme present in the system to be 110° F. or less.

16. A method of propagating at least one microorganism expressing one or more enzymes to mitigate mycotoxin contamination, wherein the method comprises:
propagating the at least one microorganism expressing one or more enzymes to mitigate mycotoxin contamination at a bioprocessing facility that produces one or more bioproducts from biomass, wherein propagating forms a propagated composition comprising the at least one microorganism expressing one or more enzymes to mitigate mycotoxin contamination; and inoculating at least a portion of the propagated composition and/or a composition derived from the propagated composition into one or more process compositions of the bioprocessing facility to mitigate mycotoxin contamination.

17. The method of any preceding embodiment, further comprising lysing the at least one microorganism expressing one or more enzymes to mitigate mycotoxin contamination to form a lysate, wherein lysing comprises mechanically and/or physically a microorganism expressing an enzyme to mitigate mycotoxin contamination to form a lysate.

18. The method of embodiment 17, wherein lysing does not include chemically lysing the at least one microorganism expressing one or more enzymes to mitigate mycotoxin contamination to form a lysate.

19. The method of any preceding embodiment, wherein the biomass comprises one or more cereal grains.

20. The method of any preceding embodiment, wherein the one or more process compositions are chosen from a feedstock composition, a milling composition, a steeping composition, a propagation composition, a saccharification composition, a fermentation composition, a stillage composition, and combinations thereof.

21. The method of any preceding embodiment, further comprising: determining if one or more mycotoxins are present in incoming grain and/or one or more process compositions of the bioprocessing facility, wherein the determining comprises measuring a concentration of the one or more mycotoxins in the incoming grain and/or the one or more process compositions of the bioprocessing facility; and propagating the at least one microorganism expressing one or more enzymes to mitigate mycotoxin contamination at the bioprocessing facility if the one or more mycotoxins are determined to be present in the incoming grain and/or the one or more process compositions of the bioprocessing facility, wherein the at least one microorganism expressing one or more enzymes to mitigate mycotoxin contamination is not propagated at the bioprocessing facility if the one or more mycotoxins are determined to not be present in the incoming grain and/or the one or more process compositions of the bioprocessing facility.

22. The method of any preceding embodiment, further comprising: determining if one or more mycotoxins are present in incoming grain and/or one or more process compositions of a different bioprocessing facility, wherein the determining comprises measuring a concentration of the one or more mycotoxins in the incoming grain and/or the one or more process compositions of the different bioprocessing facility; and propagating the at least one microorganism expressing one or more enzymes to mitigate mycotoxin contamination at the bioprocessing facility if the one or more mycotoxins are determined to be present in the incoming grain and/or the one or more process compositions of the different bioprocessing facility, wherein at least one microorganism expressing one or more enzymes to mitigate mycotoxin contamination is not propagated at the bioprocessing facility if the one or more mycotoxins are determined to not be present in the incoming grain and/or the one or more process compositions of the different bioprocessing facility.

What is claimed is:

1. A system for propagating two or more microorganisms expressing one or more enzymes to mitigate mycotoxin contamination, wherein the system comprises two or more propagation tanks, wherein each of the two or more propagation tanks comprises at least one microorganism expressing one or more enzymes to mitigate mycotoxin contamination, wherein at least one of the two or more propagation tanks comprises at least one microorganism that expresses one or more enzymes to mitigate mycotoxin contamination that is different from at least one microorganism that expresses one or more enzymes to mitigate mycotoxin contamination that is present in another propagation tank, wherein the system is configured to propagate the at least one microorganism that is present in each of the two or more propagation tanks and form a propagated composition comprising the at least one microorganism expressing one or more enzymes to mitigate mycotoxin contamination, and wherein the system is configured to couple to one or more points of a process in a bioprocessing facility to inoculate at least a portion of the propagated composition and/or a composition derived from the propagated composition from each of the two or more propagation tanks into one or more process compositions to mitigate mycotoxin contamination, wherein the bioprocessing facility is configured to produce one or more bioproducts from biomass.

2. The system of claim 1, wherein the composition derived from the propagated composition in each of the two or more propagation tanks is chosen from a concentrated propagation broth, a lysate of the at least one microorganism, a concentrated lysate of the at least one microorganism, enzyme that is isolated from a propagation broth of the at least one microorganism, enzyme that is isolated from a lysate of the at least one microorganism, and combinations thereof.

3. The system of claim 1, wherein the system is configured to be movable as a single unit among two or more bioprocessing facilities, wherein each bioprocessing facility is configured to produce one or more bioproducts from biomass.

4. The system of claim 1, wherein the each of the two or more propagation tanks has a volumetric capacity of 10,000 gallons or less.

5. The system of claim 1, wherein the each of the two or more propagation tanks has a volumetric capacity of 3,000 gallons or less.

6. The system of claim 1, wherein the two or more propagation tanks comprises three or more propagation tanks, wherein each of the three or more propagation tanks comprises at least one microorganism expressing one or more enzymes to mitigate mycotoxin contamination, wherein at least one of the three or more propagation tanks comprises at least one microorganism that expresses one or more enzymes to mitigate mycotoxin contamination that is different from at least one microorganism that expresses one or more enzymes to mitigate mycotoxin contamination that is present in another propagation tank, wherein the system is configured to propagate the at least one microorganism that is present in each of the three or more propagation tanks and form a propagated composition comprising the at least one microorganism expressing one or more enzymes to mitigate mycotoxin contamination, and wherein the system is configured to couple to one or more points of a process in a bioprocessing facility to inoculate at least a portion of the propagated composition and/or a composition derived from the propagated composition from each of the three or more propagation tanks into one or more process compositions to mitigate mycotoxin contamination, wherein the bioprocessing facility is configured to produce one or more bioproducts from biomass.

7. The system of claim 1, wherein the system is configured to remove and adjustably combine at least a portion of the contents of at least two propagation tanks in a volumetric ratio of 1:1 or different than 1:1.

8. The system of claim 1, wherein at least one of the two or more propagation tanks is configured to couple to one or more one or more points in the bioprocessing facility to receive a composition chosen from a nutrient source, a carbon source, and combinations thereof.

9. The system of claim 1, further comprising a storage system in fluid communication with at least one of the two or more propagation tanks, wherein the storage systems is configured to receive at least a portion of contents directly or indirectly from the at least one of the two or more propagation tanks, wherein the storage system comprises one or more storage containers, wherein each storage container has a capacity of 150 gallons or less.

10. The system of claim 1, further comprising a lysis system in fluid communication with at least one of the two or more propagation tanks, wherein the lysis system is configured to form a lysate with at least a portion of propagated composition from the at least one of the two or more propagation tanks.

11. The system of claim 1, further comprising a source of one or more protease inhibitors, wherein the system is configured to combine the one or more protease inhibitors with the lysate.

12. The system of claim 11, wherein the system is configured to combine the one or more protease inhibitors with the lysate at a weight ratio in a range from 1:10,000 to 1:10.

13. The system of claim 11, wherein the system is configured to combine the one or more protease inhibitors with the lysate at a weight ratio in a range from 1:10,000 to 1:1000.

14. The system of claim 1, further comprising a source of one or more stabilizers, wherein the system is configured to combine the one or more stabilizers with at least one propagated composition and/or a composition derived from the at least one propagated composition at one or more points in the system.

15. The system of claim 1, wherein the system is configured to control the temperature of the enzyme present in the system to be 110° F. or less.

16. A method of propagating two or more microorganisms expressing one or more enzymes to mitigate mycotoxin contamination using two or more propagation tanks, wherein the method comprises:
propagating at least one microorganism expressing one or more enzymes to mitigate mycotoxin contamination in a first propagation tank at a bioprocessing facility that produces one or more bioproducts from biomass, wherein propagating forms a propagated composition comprising the at least one microorganism expressing one or more enzymes to mitigate mycotoxin contamination;
propagating at least one microorganism expressing one or more enzymes to mitigate mycotoxin contamination in a second propagation tank at the bioprocessing facility that produces one or more bioproducts from biomass, wherein propagating forms a propagated composition comprising the at least one microorganism expressing one or more enzymes to mitigate mycotoxin contamination, wherein at least one microorganism expressing one or more enzymes to mitigate mycotoxin contamination that is present in the first propagation tank is different from at least one microorganism that expresses one or more enzymes to mitigate mycotoxin contamination that is present in the second propagation tank;
inoculating at least a portion of the propagated composition and/or a composition derived from the propagated composition into one or more process compositions of the bioprocessing facility to mitigate mycotoxin contamination.

17. The method of claim 16, further comprising lysing the at least one microorganism expressing one or more enzymes to mitigate mycotoxin contamination in at least one of the two or more propagation tanks to form a lysate, wherein lysing comprises mechanically and/or physically a microorganism expressing an enzyme to mitigate mycotoxin contamination to form the lysate.

18. The method of claim 17, wherein the lysing does not include chemically lysing the at least one microorganism expressing one or more enzymes to mitigate mycotoxin contamination to form the lysate.

19. The method of claim 16, wherein the biomass comprises one or more cereal grains, and wherein the bioprocessing facility is a cereal grain-to-ethanol facility.

20. The method of claim 16, wherein the one or more process compositions are chosen from a feedstock composition, a milling composition, a steeping composition, a propagation composition, a saccharification composition, a fermentation composition, a stillage composition, and combinations thereof.

21. The method of claim 16, further comprising:
determining if one or more mycotoxins are present in incoming grain and/or one or more process compositions of the bioprocessing facility, wherein the determining comprises measuring a concentration of the one or more mycotoxins in the incoming grain and/or the one or more process compositions of the bioprocessing facility; and
propagating using the two or more propagation tanks at the bioprocessing facility if the one or more mycotoxins are determined to be present in the incoming grain and/or the one or more process compositions of the bioprocessing facility, wherein propagating using the two or more propagation tanks at the bioprocessing facility is not performed if the one or more mycotoxins are determined to not be present in the incoming grain and/or the one or more process compositions of the bioprocessing facility.

22. The method of claim 16, further comprising:
determining if one or more mycotoxins are present in incoming grain and/or one or more process compositions of a different bioprocessing facility, wherein the determining comprises measuring a concentration of the one or more mycotoxins in the incoming grain and/or the one or more process compositions of the different bioprocessing facility; and
propagating using the two or more propagation tanks at the bioprocessing facility if the one or more mycotoxins are determined to be present in the incoming grain and/or the one or more process compositions of the different bioprocessing facility, wherein propagating using the two or more propagation tanks at the bioprocessing facility is not performed if the one or more mycotoxins are determined to not be present in the incoming grain and/or the one or more process compositions of the different bioprocessing facility.

23. A method of propagating at least one microorganism expressing one or more enzymes to mitigate mycotoxin contamination, wherein the method comprises:
propagating the at least one microorganism expressing one or more enzymes to mitigate mycotoxin contamination at a bioprocessing facility that produces one or more bioproducts from biomass, wherein propagating forms a propagated composition comprising the at least one microorganism expressing one or more enzymes to mitigate mycotoxin contamination;
lysing the at least one microorganism expressing one or more enzymes to mitigate mycotoxin contamination in the propagated composition to form a lysate, wherein lysing comprises mechanically and/or physically the at least one microorganism expressing one or more enzymes to mitigate mycotoxin contamination in the propagated composition to form the lysate; and
inoculating at least a portion of the lysate into one or more process compositions of the bioprocessing facility to mitigate mycotoxin contamination.
